(19) United States
(12) Patent Application Publication
(10) Pub. No.: US 2014/0194832 A1
(43) Pub. Date: Jul. 10, 2014

(54) INSTILLATOR FOR INJECTION OF RINGER'S SOLUTION

(71) Applicant: HUBIOMED Co., Ltd., Seoul (KR)

(72) Inventor: Sun CHUNG, Seongnam-si (KR)

(73) Assignee: HUBIOMED Co., Ltd., Seoul (KR)

(21) Appl. No.: 14/096,393

(22) Filed: Dec. 4, 2013

(30) Foreign Application Priority Data
Jan. 4, 2013 (KR) 10-2013-0001186
Oct. 16, 2013 (KR) 10-2013-0122990

(51) Int. Cl. A61J 1/14; A61M 5/168

(52) U.S. Cl. CPC A61J 1/1475; A61M 5/16881
USPC 604/248

(57) ABSTRACT

An instillator for injection of a Ringer's solution temporarily stores Ringer's solutions or others, and couples a precise flow regulator onto an instillation container, thereby allowing the solutions to be administered more accurately, preventing accidents from happening because the regulator is out of a patient's reach, and allowing a connected tube to be easily arranged. The instillator includes an instillation container, a solution inflow tube with a penetration needle and a solution guide passage, and a lower stopper with a solution drainage passage and a tube connector, wherein a drainage guide plate with a drainage hole is inserted into the container to discharge a solution downwardly, a precise flow regulator is coupled onto the plate to be rotated from side to side, and an instillation hole communicating with the tube connector is formed in the regulator.

### BACKGROUND OF THE INVENTION

1. Technical Field

The present invention relates to an instillator for injection of a Ringer's solution, and especially provides a novel instillator for injection of a Ringer's solution which temporarily stores solutions falling from bottles of Ringer's solution or others, and integrally couples a precise flow regulator for regulating instillation rates of the solutions onto the bottom of an instillation container for instilling and feeding the stored solutions into a blood vessel via a tube, thereby allowing the solutions to be administered more accurately, preventing unexpected accidents from happening because the regulator is disposed in a position where a patient cannot reach, and allowing the length of a connected tube to be easily arranged.

2. Description of the Related Art

A solution is generally administered to a patient using a solution feed set after a surgical operation.

The solution feed set, which is used in a hospital as a type of medical equipment, consists of a solution bottle which is sealed by a rubber stopper in a state in which a solution is stored therein, an instillation container which has an entry needle formed on the top thereof to penetrate the rubber stopper closing the solution bottle, includes a solution drainage tube formed inside the entry needle in a hollow manner so that the solution stored in the solution bottle is discharged, and temporarily stores the solution discharged via the solution drainage tube in the lower portion of the instillation container as well as instills the solution drop by drop, a vein needle which is connected to the bottom of the instillation container through a tube and allows the solution falling from the instillation container to flow into a blood vessel, and a regulator which is inserted into the middle of the tube and regulates an amount of the solution falling from the instillation container and flowing through the tube.

When using the solution feed set with such a configuration, firstly, a user completes closes the regulator to block an air flow between the instillation container and the vein needle, and then vertically inserts the entry needle into the rubber stopper on the solution bottle, and after that, immediately turns the solution bottle upside down and lifts up and hangs the bottle.

After that, the user slightly presses the instillation container to fill the container with the solution about half and completely opens the regulator to remove air in the tube, and then re-closes the regulator and fixedly inserts the vein needle into a blood vessel, and after that, injects the solution into the blood vessel by controlling an injection amount of the solution using the regulator.

However, the above-mentioned regulator of the conventional solution feed set is configured in such a manner that a saw-toothed disk is disposed to be vertical against the tube and moves upwardly and downwardly to compress the tube, and therefore the user decides an injection amount of the solution at random, an injection amount of the solution (instillation rate) may not be set as much as the user desires, because the elasticity of the tube causes the saw-toothed disk to slightly move even though the tube is closed, and a Flow Regulator, which is used as an additional product, has to be additionally attached to the middle of the tube connected between the instillation container and the vein needle so that a required amount of the solution is injected.

In this case, the tube is connected in a complicated and long manner, and therefore a longer time is taken to inject the solution from the instillation container to the vein needle, inconvenience is caused when a patient moves, and unexpected situations may happen because the regulator is attached to a position in which the patient may adjust an injection amount of the solution by mistake or on his or her own way.

### SUMMARY OF THE INVENTION

The present invention is applied to solve the above-mentioned disadvantages of the prior art, and an objective of the present invention is to provide a novel instillator for injection of a Ringer's solution which temporarily stores solutions falling from bottles of Ringer's solution or others, and integrally couples a precise flow regulator for regulating instillation rates of the solutions onto the bottom of an instillation container for instilling and feeding the stored solutions into a blood vessel via a tube, thereby allowing the solutions to be administered more accurately, and preventing unexpected accidents from happening because the regulator is disposed in a position where a patient cannot reach.

Another objective of the present invention is to provide a novel instillator for injection of a Ringer's solution which allows a user to easily regulate an instillation rate of the solution, thereby enhancing convenience in use and improving safety.

The objectives are achieved by an instillator for injection of a Ringer's solution according to the present invention including: a cylindrical instillation container which is made out of a transparent material; a solution inflow tube which is formed on the top of the instillation container in a projecting manner, has a penetration needle, which penetrates a rubber stopper for closing a solution bottle, formed on the top of the solution inflow tube, and has a solution guide passage, which guides the solution in the solution bottle to flow into the instillation container, formed along a vertical centerline of the solution inflow tube; and a lower stopper which is inserted into the bottom of the instillation container, has a solution drainage passage, which is used to discharge the solution stored in the instillation container, formed on the top surface of the lower stopper in a penetrating manner so that the passage reaches the bottom surface of the lower stopper, and has a tube connector formed on the bottom of the solution drainage passage in a projecting manner; characterized in that a drainage guide plate with a drainage hole is fixedly inserted into the bottom of the instillation container to downwardly discharge the solution guided into the lower portion of the instillation container, a precise flow regulator is coupled onto the outer circumference of the drainage guide plate to be rotated from side to side, and an instillation hole communicating with the tube connector is formed on the inner surface of the bottom of the precise flow regulator.

The instillator for injection of a Ringer's solution according to the present invention is characterized in that a solution guide plate for guiding the solution falling via the solution inflow tube to flow towards the solution drainage passage is inclinedly formed over the range from an upper position on the inner circumference of one lateral side of the instillation container to a position adjacent to the solution drainage passage formed on the top surface of the lower stopper.

### BRIEF DESCRIPTION OF DRAWINGS

FIG. 1 illustrates an instillator for injection of a Ringer's solution according to the present invention in a perspective view;

FIG. 2 illustrates the interior of the instillator for injection of a Ringer's solution according to the present invention in a perspective view;

FIG. 3 illustrates the instillator for injection of a Ringer's solution according to the present invention in a sectional view;

FIG. 4 illustrates a state in which the instillator for injection of a Ringer's solution according to the present invention is used in a sectional view;

FIG. 5 illustrates the instillator for injection of a Ringer's solution according to the present invention in an exploded perspective view;

FIG. 6 illustrates a state in which a precise flow regulator of the instillator for injection of a Ringer's solution according to the present invention is coupled in a top view; and

FIG. 7 illustrates an operation for regulating an instillation rate of a solution using the precise flow regulator of the instillator for injection of a Ringer's solution according to the present invention in a top view.

### DETAILED DESCRIPTION

Hereinafter, a preferred embodiment of the present invention is described in more detail with reference to the accompanying drawings.

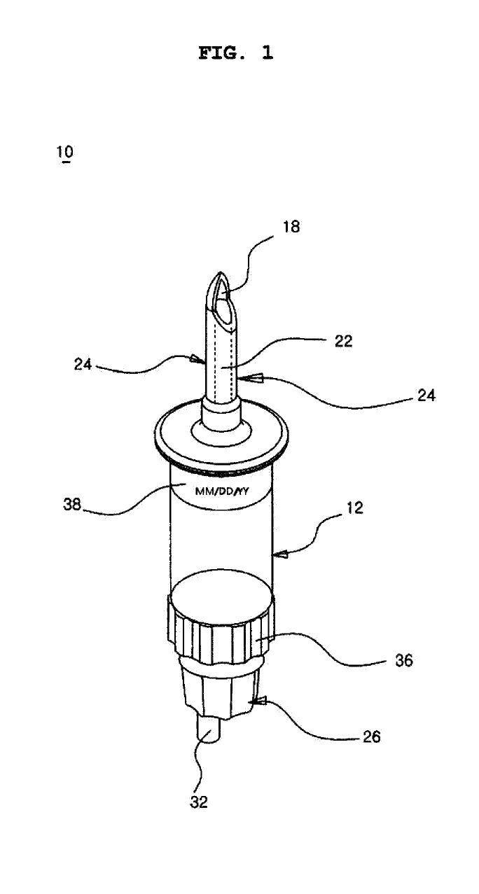

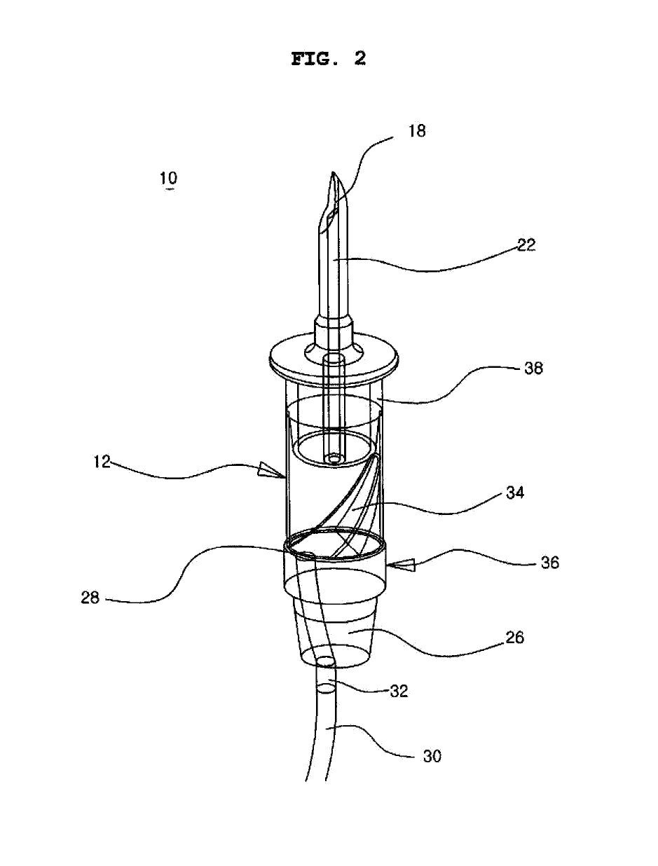

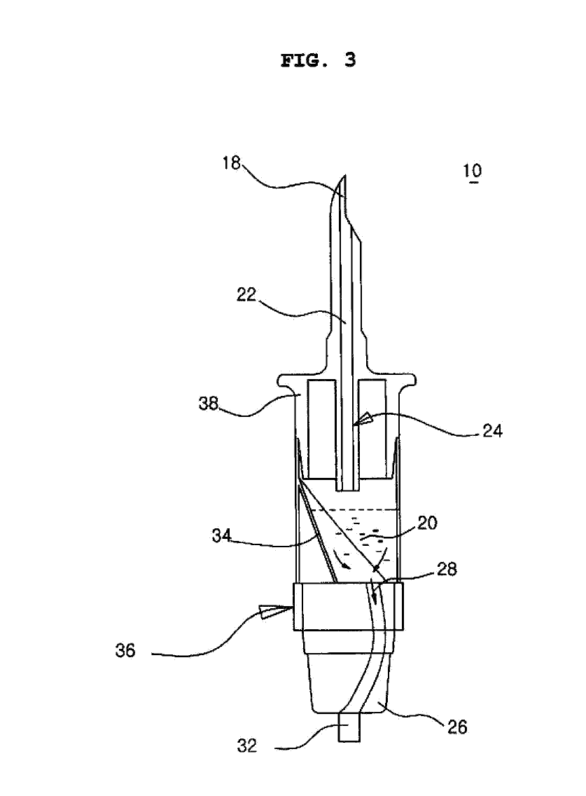

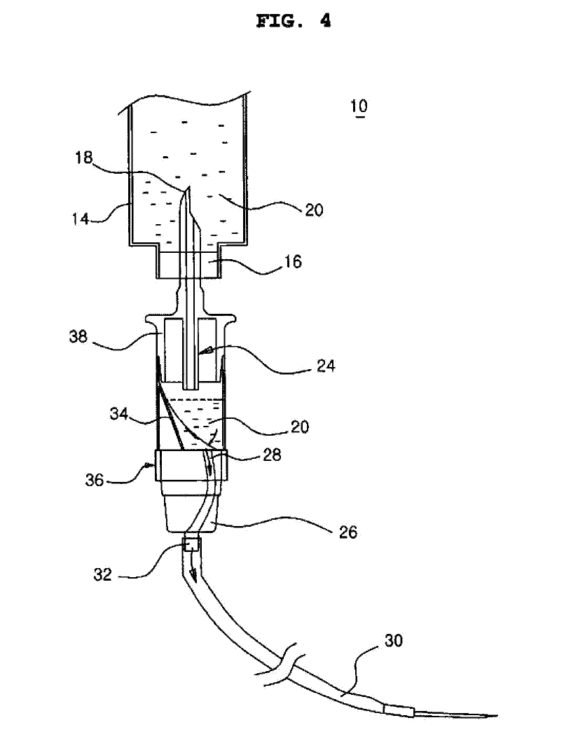

Referring to FIGS. 1 through 7, an instillator for injection of a Ringer's solution 10 according to the present invention includes a cylindrical instillation container 12 made out of a transparent material.

The instillation container 12 has a solution inflow tube 24 outwardly formed on the top thereof in a projecting manner, wherein the solution inflow tube consists of a penetration needle 18, which penetrates a rubber stopper 16 for closing a solution bottle 14, formed on the top of the solution inflow tube, and a solution guide passage 22, which guides a solution 20 in the solution bottle 14 to flow into the instillation container 12, formed along a vertical centerline of the solution inflow tube.

In addition, the instillation container 12 has an additional lower stopper 26 inserted into the bottom thereof, wherein the lower stopper 26 has a solution drainage passage 28, which is used to discharge the solution 20 filling the instillation container 12, formed on the top surface of the lower stopper in a penetrating manner so that the solution drainage passage reaches the bottom surface of the lower stopper.

Furthermore, the solution drainage passage 28 has a tube connector 32, which insertably receives an additional tube 30 with a vein needle connected to an end of the tube, formed on the bottom thereof in a projecting manner.

According to the present invention, a solution guide plate 34 for guiding the solution 20 falling via the solution inflow tube 24 to flow towards the solution drainage passage 28 is inclinedly formed over the range from an upper position on the inner circumference of one lateral side of the instillation container 12 to a position adjacent to the solution drainage passage 28 formed on the top surface of the lower stopper 26.

The lower stopper 26 has a precise flow regulator 36, which regulates an instillation rate of the solution 20 falling via the solution drainage passage 28 by means of a left or right rotation of the precise flow regulator, insertably coupled onto the outer circumference of the lower stopper.

According to the present invention with such a configuration, the instillation container 12 may have a date marking portion 38 on which a replacement date is marked formed on the outer circumference of the upper portion thereof.

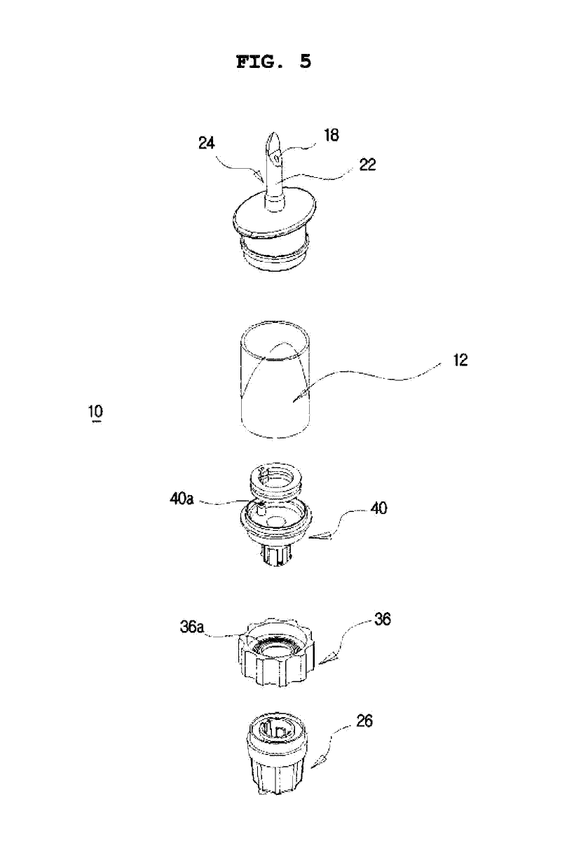

Besides, as shown in FIGS. 5 through 7, to regulate an instillation rate of the solution 20, a drainage guide plate 40 which includes a drainage hole 40a and is used to downwardly discharge the solution 20 guided into the lower portion of the instillation container 12 is fixedly inserted into the bottom of the instillation container 12, that is, a portion below the solution guide plate 34, the precise flow regulator 36 is coupled onto the outer circumference of the drainage guide plate 40 to be rotated from side to side, and an instillation hole 36a communicating with the tube connector 32 is formed on the inner surface of the bottom of the precise flow regulator 36.

An operation of the present invention with such a configuration is as follows.

Referring to FIGS. 1 through 4, when feeding a solution to a patient using an instillator for injection of a Ringer's solution 10 according to the present invention, firstly, a user hangs a solution bottle 14 upside down, and then inserts a penetration needle 18 on the tip of a solution inflow tube 24 outwardly protruding from the top of an instillation container 12 into a rubber stopper 16 of the solution bottle 14 so that the penetration needle penetrates the rubber stopper.

Here, the solution 20 stored in the solution bottle 14 falls into the instillation container 12 via a solution guide passage 22 formed in the solution inflow tube 24, and the solution 20 falling from the bottom of the solution inflow tube 24 flows towards a solution drainage passage 28 formed on the top surface of a lower stopper 26 along an inclined solution guide plate 34 in the instillation container 12.

Also, the solution 20 flowing towards the solution drainage passage 28 is instilled along the solution drainage passage 28, and then flows into a blood vessel via a tube 30 connected to a tube connector 32 as well as a vein needle coupled to the tip of the tube 30.

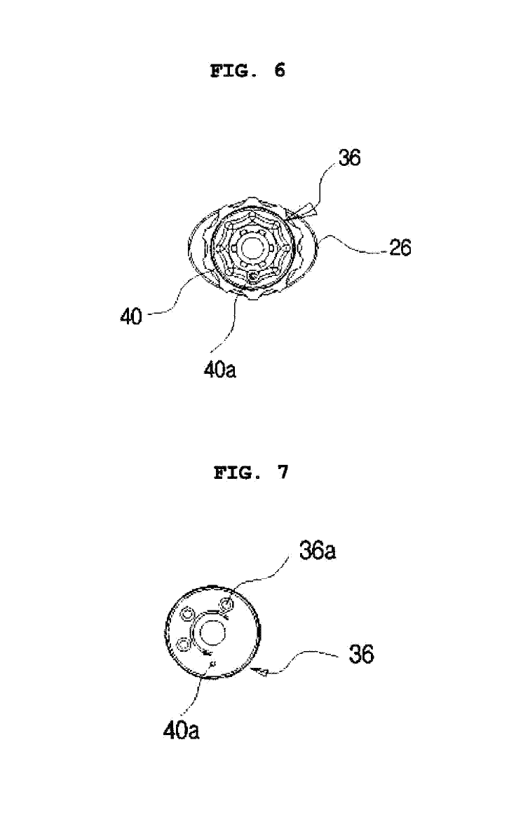

Here, when regulating an instillation rate of the solution 20 instilled via the solution drainage passage 28, as shown in FIGS. 6 and 7, rotating a precise flow regulator 36 coupled to the outer circumference of the upper portion of the lower stopper 26 to the left or right allows an instillation hole 36a formed on the inner surface of the bottom of the precise flow regulator 36 to move close to or far from a drainage hole 40a on the drainage guide plate 40, wherein an instillation rate of the solution 20 is increased when the instillation hole 36a moves closer to the drainage hole 40a on the drainage guide plate 40, while an instillation rate of the solution 20 is decreased when the instillation hole 36a moves farther from the drainage hole 40a on the drainage guide plate 40.

Besides, when a date marking portion 38 is formed on the outer circumference of the upper portion of the instillation container 12, a replacement date may be easily marked, and therefore the solution 20 is safely and conveniently fed.

According to the present invention, an instillator for injection of a Ringer's solution has an advantage that the instillator temporarily stores solutions falling from bottles of Ringer's solution or others, and integrally couples a precise flow regulator for regulating instillation rates of the solutions onto the bottom of an instillation container for instilling and feeding the stored solutions into a blood vessel via a tube, thereby allowing the long tube connected to the solution bottle to be easily arranged in a hospital room.

In addition, the instillator has another advantage that a user easily regulates an instillation rate of the solution flowing into the instillation container, so that convenience in use is enhanced and safety is improved.

What is claimed is:

1. Instillator for injection of a Ringer's solution, comprising:
a cylindrical instillation container which is made out of a transparent material;
a solution inflow tube which is formed on the top of the instillation container in a projecting manner, has a penetration needle, which penetrates a rubber stopper for closing a solution bottle, formed on the top of the solution inflow tube, and has a solution guide passage, which guides a solution stored in the solution bottle to flow into the instillation container, formed along a vertical centerline of the solution inflow tube; and
a lower stopper which is inserted into the bottom of the instillation container, has a solution drainage passage, which is used to discharge the solution filling the instillation container, formed on the top surface of the lower stopper in a penetrating manner so that the solution drainage passage reaches the bottom surface of the lower stopper, and has a tube connector formed on the bottom of the solution drainage passage in a projecting manner,
wherein a drainage guide plate with a drainage hole is fixedly inserted into the bottom of the instillation container to downwardly discharge the solution guided into the lower portion of the instillation container,
a precise flow regulator is coupled onto the outer circumference of the drainage guide plate to be rotated from side to side, and
an instillation hole communicating with the tube connector is formed on the inner surface of the bottom of the precise flow regulator.

2. The instillator for injection of a Ringer's solution according to claim 1, wherein a solution guide plate for guiding the solution falling via the solution inflow tube to flow towards the solution drainage passage is inclinedly formed over the range from an upper position on the inner circumference of one lateral side of the instillation container to a position adjacent to the solution drainage passage formed on the top surface of the lower stopper.

3. The instillator for injection of a Ringer's solution according to claim 1, wherein the instillation container has a date marking portion on which a replacement date is marked formed on the outer circumference of the upper portion thereof.
